(19) United States
(12) Patent Application Publication
(10) Pub. No.: US 2025/0078266 A1
(43) Pub. Date: Mar. 6, 2025

(54) MEDICAL IMAGE PROCESSING APPARATUS, MEDICAL IMAGE PROCESSING METHOD, AND STORAGE MEDIUM

(71) Applicant: CANON MEDICAL SYSTEMS CORPORATION, Otawara-shi (JP)

(72) Inventors: Kyotaro HORIO, Nasushiobara (JP); Takahiko NISHIOKA, Otawara (JP); Shiro KATO, Utsunomiya (JP)

(73) Assignee: CANON MEDICAL SYSTEMS CORPORATION, Otawara-shi (JP)

(21) Appl. No.: 18/816,223

(22) Filed: Aug. 27, 2024

(30) Foreign Application Priority Data
Aug. 28, 2023 (JP) 2023-138119
Aug. 8, 2024 (JP) 2024-132757

(51) Int. Cl. G06T 7/00; G06T 7/13; G06T 7/246; G06T 11/00

(52) U.S. Cl. CPC G06T 7/0012; G06T 7/13; G06T 7/246; G06T 11/001; G06T 2207/30104; G06T 2210/41

(57) ABSTRACT

A medical image processing apparatus includes processing circuitry configured to: acquire medical image data of a site including a first blood vessel that is an artery and a second blood vessel that is an artery or a vein; specify a dominant area of the first blood vessel based on the medical image data, a blood flow strength coefficient and a damping coefficient of the first blood vessel, and a blood flow strength coefficient and a damping coefficient of the second blood vessel; and output data for displaying the specified dominant area.

### CROSS REFERENCE TO RELATED APPLICATION

This application is based upon and claims the benefit of priority from the prior Japanese Patent Application No. 2023-138119, filed on Aug. 28, 2023, and the prior Japanese Patent Application No. 2024-132757, filed on Aug. 8, 2024, the entire contents of which are incorporated herein by reference.

### FIELD

The embodiments disclosed in the present specification and in the drawings relate to a medical image processing apparatus, a medical image processing method, and a storage medium.

### BACKGROUND

An area supplied with nutrients by blood that diffuses from an artery is called a dominant area (or a blood flow area) and represents an important indicator in vascular treatment. For example, after treating a coronary artery disease, a dominant area of the treated coronary artery is specified and an area that can be subjected to reperfusion is comprehended from the specified dominant area. Therefore, a dominant area is an important indicator when estimating an effect of treatment.

Conventionally, Voronoi tessellation is known as a method of specifying a dominant area using a medical image. Voronoi tessellation is a method of partitioning regions by determining to which of a plurality of seeds (generators) arranged at arbitrary positions in a metric space another point in the same metric space is closest. For example, a dominant area of a selected coronary artery is specified by area expansion based on a shape of the coronary artery. A borderline between dominant areas constitutes a part of a perpendicular bisector of two seeds. However, in the case of this method, since a dominant area is specified by only taking distances from seeds in spatial coordinates into consideration, it is difficult to accurately specify a dominant area for each individual patient.

### BRIEF DESCRIPTION OF THE DRAWINGS

FIG. 1 is a diagram showing an example of a configuration of a medical image processing apparatus according to a first embodiment;

FIG. 2A is a diagram for illustrating an example of a factor that determines a damping coefficient of a blood flow;

FIG. 2B is a diagram for illustrating an example of a factor that determines a damping coefficient of a blood flow;

FIG. 2C is a diagram for illustrating an example of a factor that determines a damping coefficient of a blood flow;

FIG. 3 is a flowchart for illustrating an example of a medical image processing method according to the first embodiment;

FIG. 4 is a flowchart for illustrating an example of a specification method of a dominant area according to the first embodiment;

FIG. 5 is a diagram showing an example of a vector field constituted of blood flow vectors of arteries;

FIG. 6 is a diagram for illustrating a specification method of a dominant area according to the first embodiment;

FIG. 7 is a diagram showing an example of dominant areas specified by a boundary when arteries are present on a surface of tissue;

FIG. 8 is a flowchart for illustrating an example of a specification method of a dominant area according to a modification of the first embodiment;

FIG. 9A is a diagram for illustrating the specification method of a dominant area according to the modification of the first embodiment;

FIG. 9B is a diagram showing an example of a vector field constituted of blood flow vectors of a vein;

FIG. 10 is a diagram showing an example of a screen on which lines depicting blood flows are superimposed and displayed on a medical image;

FIG. 11 is a diagram showing an example of a screen on which dominant areas are displayed by gradation;

FIG. 12 is a diagram showing an example of a display image in which a heart surface is developed into a two-dimensional image by equal-area map projection;

FIG. 13 is a diagram showing an example of a display image after a thrombus has been removed by simulation;

FIG. 14A is a diagram showing an image example on which a relatively-bold borderline is superimposed and displayed when a diameter of an artery is relatively large;

FIG. 14B is a diagram showing an image example on which a relatively-fine borderline is superimposed and displayed when a diameter of an artery is relatively small;

FIG. 15 is a diagram showing an image example in which a boundary is displayed by gradation in accordance with strength of blood flows toward the boundary;

FIG. 16 is a diagram showing an image example in which a pop-up message is displayed on a medical image;

FIG. 17 is a diagram showing an image example in which a pop-up message is displayed on a medical image;

FIG. 18 is a flowchart for illustrating an example of a medical image processing method according to a second embodiment;

FIG. 19 is a flowchart for illustrating a specification method of a dominant area according to the second embodiment;

FIG. 20 is a diagram for illustrating the specification method of a dominant area according to the second embodiment;

FIG. 21 is a diagram showing an example of a configuration of a medical image processing apparatus according to a third embodiment;

FIG. 22 is a flowchart for illustrating an example of a medical image processing method according to a third embodiment; and

FIG. 23 is a diagram showing an example of a screen according to the third embodiment.

### DETAILED DESCRIPTION

Hereinafter, medical image processing apparatuses according to first to third embodiments will be described with reference to the drawings. In the following description, constituent elements with substantially a same function and a same configuration will be denoted by a same reference sign and overlapping descriptions will only be given when necessary. In addition, terms (parallel, orthogonal, and the like) indicating a geometric condition used in the present specification are not confined to strict definitions and are to be interpreted to include an extent to which similar functions can be expected.

### First Embodiment

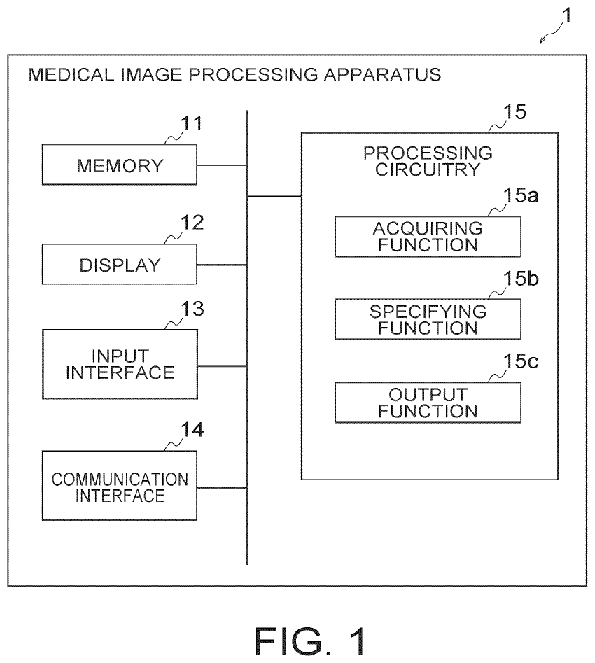

FIG. 1 shows an example of a configuration of a medical image processing apparatus 1 according to a first embodiment.

As will be described in detail below, the medical image processing apparatus 1 according to the present embodiment is configured to acquire medical image data of a site including a plurality of arteries from an external medical image diagnostic apparatus, an external medical image storage server, or the like (not illustrated), specify a dominant area of each artery by taking a blood flow strength coefficient (R) and a damping coefficient (β) of each artery into consideration, and output data for displaying the specified dominant area.

As shown in FIG. 1, the medical image processing apparatus 1 includes a memory 11, a display 12, an input interface 13, a communication interface 14, and processing circuitry 15. Details of each component will be described hereinafter.

The memory 11 is connected to the processing circuitry 15 and stores various kinds of information to be used by the processing circuitry 15. The memory 11 is realized by, for example, a semiconductor memory element such as a RAM (Random Access Memory) or a flash memory, a hard disk, an optical disk, or the like. The memory 11 stores various kinds of programs necessary for the processing circuitry 15 to execute respective functions, various kinds of data to be processed by the programs, and the like. Note that the various kinds of data to be handled in the present specification are typically digital data.

The display 12 displays various kinds of images and information. For example, the display 12 displays medical images and GUIs (Graphical User Interfaces) for accepting user operations. In the present embodiment, for example, the display 12 is constituted of a liquid crystal display, a CRT (Cathode Ray Tube) display, or the like.

The input interface 13 accepts various kinds of input operations, converts the accepted input operations into electric signals, and outputs the electric signals to the processing circuitry 15. For example, the input interface 13 is realized by a mouse and a keyboard, a touch panel, a trackball, a manual switch, a foot switch, a button, a joystick, or the like. The input interface 13 is not limited to the physical operating members described above and may be a circuit which receives a signal corresponding to an input operation from an external input device provided separately from the medical image processing apparatus 1 and which outputs the signal to the processing circuitry 15.

The communication interface 14 communicates with other apparatuses (a medical image diagnostic apparatus, a medical image storage server, and the like) via an in-hospital network or an external communication network such as the Internet according to various communication protocols.

The processing circuitry 15 is arithmetic circuitry that performs various kinds of arithmetic operations and controls operations of the medical image processing apparatus 1. As shown in FIG. 1, the processing circuitry 15 includes an acquiring function 15a, a specifying function 15b, and an output function 15c. The acquiring function 15a is an example of an acquirer, the specifying function 15b is an example of a specifier, and the output function 15c is an example of an outputter.

In the present embodiment, the respective processing functions executed by the acquiring function 15a, the specifying function 15b, and the output function 15c are stored in the memory 11 in the form of programs that can be executed by a computer. Specifically, the processing circuitry 15 is constituted of a processor and realizes a function corresponding to each program by reading and executing the program from the memory 11. In other words, the processing circuitry 15 in a state of having read each program is to include each function shown in the processing circuitry 15 in FIG. 1.

While FIG. 1 shows a case where the respective processing functions of the acquiring function 15a, the specifying function 15b, and the output function 15c are realized by the single processing circuitry 15, the embodiment is not limited thereto. For example, the processing circuitry 15 may be configured as a combination of a plurality of independent processors and each processing function may be realized by having each processor execute each program. In addition, each processing function included in the processing circuitry 15 may be realized by being appropriately integrated to a single processing circuitry or distributed among a plurality of processing circuitries.

Next, details of each processing function will be described.

The acquiring function 15a acquires medical image data of a subject (a patient, an examinee, or the like) from an external apparatus (such as an electronic health record system) or the memory 11. For example, the medical image data is volume data such as an MRI image, a CT image, or the like.

The medical image data is data of a medical image of a site including an artery (a first blood vessel) and an artery or a vein (a second blood vessel). In the present embodiment, the medical image data is data of a medical image of a site including a plurality of arteries. In other words, the first blood vessel and the second blood vessel are both arteries.

Although the site is not particularly limited, for example, the site is the heart, the liver, or the brain. When the site is the heart, the medical image data includes data of a coronary artery that runs on a surface of a tissue (myocardium). In addition, when the site is the liver or the brain, the medical image data includes data related to an artery inside a tissue.

Note that the acquiring function 15a may acquire information other than medical image data such as data of blood pressure or the like of a subject.

The specifying function 15b is configured to specify at least a dominant area of the first blood vessel based on the medical image data, a blood flow strength coefficient and a damping coefficient of the first blood vessel, and a blood flow strength coefficient and a damping coefficient of the second blood vessel.

In the present embodiment, the specifying function 15b is configured to specify dominant areas of the first and second arteries based on the medical image data, a blood flow strength coefficient and a damping coefficient of the first artery, and a blood flow strength coefficient and a damping coefficient of the second artery. The specifying function 15b specifies the dominant areas of the first and second arteries by demarcating a boundary between the dominant area of the first artery and the dominant area of the second artery. In this case, the dominant area of an artery is an area supplied with nutrients by the artery and is also called a blood flow area. The boundary is not limited to a borderline and may be a boundary surface.

The blood flow strength coefficient of an artery is a coefficient that represents a strength of a blood flow of blood diffused from inside to outside of an artery. In other words, the blood flow strength coefficient is a coefficient that represents a force by which blood inside the artery passes through the vascular wall and is diffused to peripheral tissue.

Specifically, the blood flow strength coefficient of an artery is determined by form factors and functional factors of the artery. Examples of form factors include a vascular diameter, a vascular length, a vascular position, a thickness of a vascular wall, and the like of the artery. Examples of functional factors include blood pressure and blood flow. The blood flow strength coefficient increases as the vascular diameter increases. In addition, the blood flow strength coefficient increases as the vascular length increases. The blood flow strength coefficient also increases when blood pressure or blood flow is high. For example, the vascular position indicates a distance of the artery from the heart and the shorter the distance from the heart, the larger the blood flow strength coefficient. Since the thicker the vascular wall, the less readily blood is diffused to outside of the blood vessel, the blood flow strength coefficient decreases. As described above, the blood flow strength coefficient of an artery according to the present embodiment is a coefficient based on at least one of the vascular diameter, the vascular length, the vascular position, the thickness of the vascular wall, blood pressure, and blood flow of the artery. The specifying function 15b determines the blood flow strength coefficient based on these factors.

Information related to the form factors and the functional factors described above may be obtained from medical image data or from an external device such as a sphygmomanometer. For example, information on form factors is acquired from medical image data and information on functional factors is acquired from an external device. Note that information on functional factors can also be acquired from medical image data. For example, a vascular shape may be acquired from a CT image, blood pressure and blood flow may be obtained by fluid calculation, or blood flow may be obtained from an MRI image.

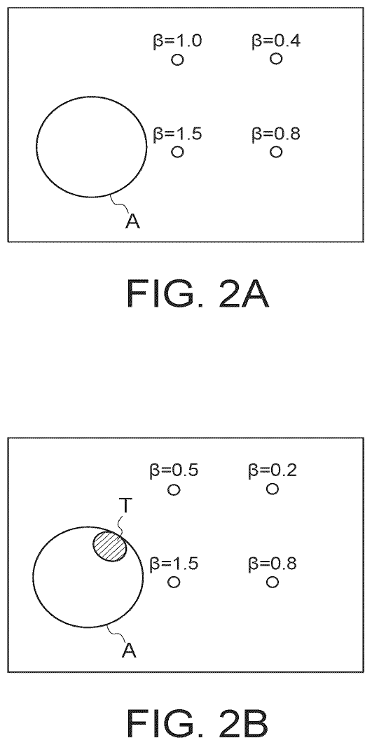

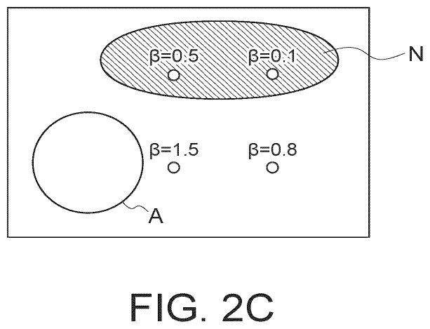

The damping coefficient of an artery is a coefficient that represents damping characteristics of blood flow in a peripheral tissue (such as the myocardium) of the artery. Specifically, the damping coefficient of an artery is determined based on form factors and functional factors of the artery and the peripheral tissue of the artery. Examples of form factors include a distance from the artery and a position of a thrombus inside the artery. As shown in FIG. 2A, the farther from an artery A, the smaller the value of the damping coefficient β (in other words, the larger the damping of blood flow). As shown in FIG. 2B, when there is a thrombus T in the artery A, the damping coefficient β decreases in a direction where the thrombus T is present. Examples of functional factors include properties of the peripheral tissue of the artery. For example, when a part of the peripheral tissue of the artery A has become necrotic and sclerotic due to a disease, blood is less readily diffused and the damping coefficient decreases. In FIG. 2C, since blood is less readily diffused in a tissue N in which ischemia has worsened and the tissue N has hardened, the damping coefficient β decreases. The damping coefficient β according to the present embodiment is a coefficient based on at least one of a distance from an artery, a position of a thrombus inside the artery, and properties of tissue surrounding the artery. The specifying function 15b determines the damping coefficient based on these factors. While the circular marks in FIGS. 2A to 2C only depict representative points in a peripheral area of an artery, the damping coefficient may be set for each data point (voxel) of the medical image data.

The properties of the tissue may be characteristics (characteristics that affect diffusion of blood) of the tissue other than hardness. A portion of which properties differ from other portions such as a necrotic zone or the tissue N described above can be detected by CT Perfusion in which a blood flow of a tissue is measured using a contrast agent. Alternatively, the portion may be detected by MRI Perfusion. Otherwise, a portion with different properties may be detected by studying a function of a tissue by a strain analysis using a result of ultrasonography.

In addition, besides specifying a dominant area based on the blood flow strength coefficient and the damping coefficient of a present artery (artery prior to treatment), the specifying function 15b may specify a dominant area based on the blood flow strength coefficient and the damping coefficient of a future artery (artery after treatment). In other words, the specifying function 15b may specify a dominant area based on the blood flow strength coefficient and the damping coefficient of an artery from which a thrombus has been removed. Furthermore, in doing so, the dominant area of the artery after a virtual treatment may be simulated using virtual values as the blood flow strength coefficient and the damping coefficient.

The output function 15c outputs data (display data) for displaying the dominant area specified by the specifying function 15b. In the present embodiment, the output function 15c outputs data for displaying the dominant areas of the first and second arteries specified by the specifying function 15b. The output function 15c transmits the display data to the display 12. The display 12 having received the display data displays an image created by superimposing and displaying a borderline dividing the dominant areas of the first and second arteries on a medical image in which the respective arteries are drawn. Note that the output function 15c may transmit the display data via the communication interface 14 to an external display apparatus (not illustrated) connected to the medical image processing apparatus 1.

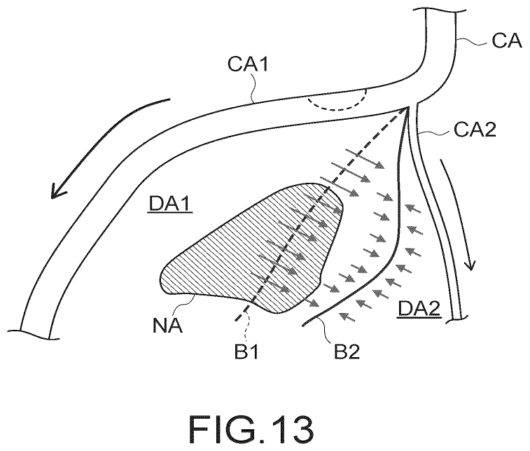

When a dominant area of an artery after a virtual treatment is specified, the output function 15c may output data for displaying a dominant area (first dominant area) of the artery prior to treatment and output data for displaying the dominant area (second dominant area) of the artery after the virtual treatment. In this case, the output function 15c may output data for comparably displaying the first dominant area and the second dominant area. For example, the output function 15c outputs data for displaying an image including the first dominant area and an image including the second dominant area side by side on a same screen. Alternatively, the output function 15c may superimpose and display a boundary defining the first dominant area and a boundary defining the second dominant area on a same medical image (refer to FIG. 13 to be described later).

### <Medical Image Processing Method (First Embodiment)>

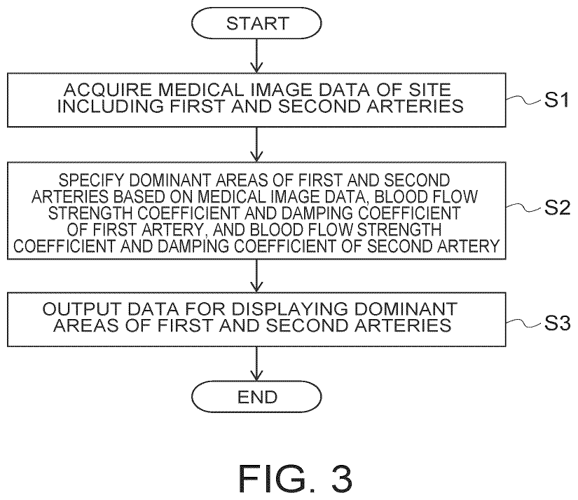

Next, an example of a medical image processing method using the medical image processing apparatus 1 described above will be described with reference to the flowchart in FIG. 3.

Step S1: The acquiring function 15a acquires medical image data of a site including first and second arteries. In the present step, for example, the acquiring function 15a acquires medical image data of a subject from an image storage apparatus (for example, a reconstruction server that reconstructs a diagnostic image from imaged data or a PACS (Picture Archiving and Communication System)) that is communicably connected to the medical image processing apparatus 1.

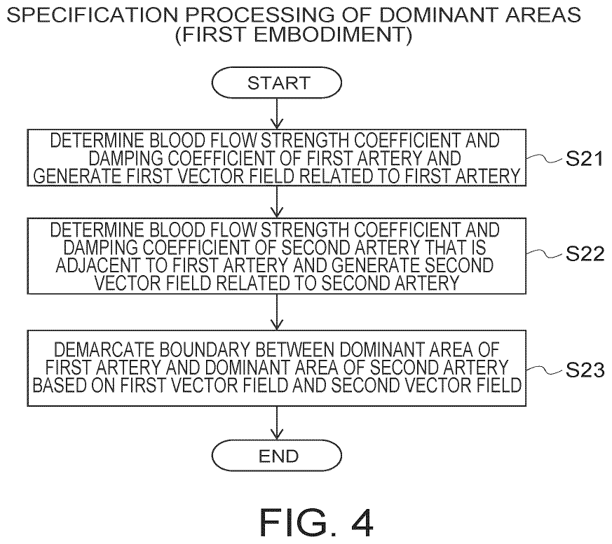

Step S2: The specifying function 15b specifies dominant areas of a plurality of arteries based on the medical image data acquired in step S1, a blood flow strength coefficient and a damping coefficient of the first artery, and a blood flow strength coefficient and a damping coefficient of the second artery. The present step will be described in detail with reference to the flowchart in FIG. 4.

Step S21: The specifying function 15b determines the blood flow strength coefficient and the damping coefficient of the first artery and generates a first vector field related to the first artery. In the present step, for example, attention is focused on one artery among the plurality of arteries included in the medical image data acquired in the previous step S1 and a vector field of the artery of attention is generated. First, the specifying function 15b determines the blood flow strength coefficient based on at least one of a diameter, a length, a position, the thickness of a wall, blood pressure, and blood flow of the first artery. Furthermore, the specifying function 15b determines the damping coefficient based on at least one of a distance from the first artery, a position of a thrombus, and properties of tissue surrounding the first artery. In addition, the specifying function 15b generates a first vector field (distance field) related to the first artery based on the determined blood flow strength coefficient and the determined damping coefficient.

A blood flow vector constituting the first vector field has a direction of diffusion from the first artery and a magnitude in accordance with the blood flow strength coefficient and the damping coefficient of the first artery. For example, the blood flow vector has a magnitude proportional to a product of the blood flow strength coefficient and the damping coefficient.

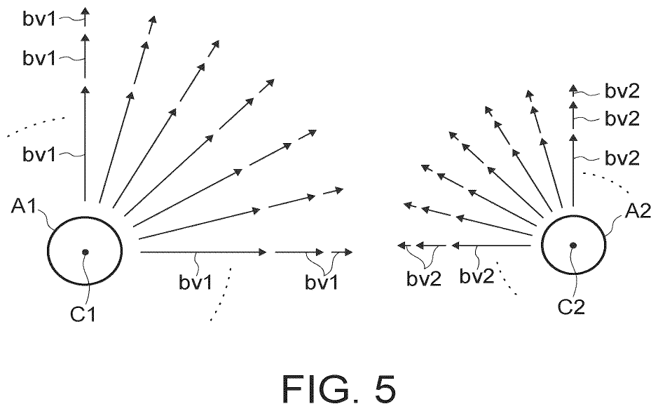

FIG. 5 shows an example of a vector field constituted of blood flow vectors of arteries. An artery A1 on a left side of FIG. 5 is an example of the first artery. A plurality of blood flow vectors bv1 that radially spread from a center C1 of the artery A1 constitute the first vector field. Each blood flow vector bv1 has a direction of diffusion from the artery A1 and a magnitude in accordance with the blood flow strength coefficient and the damping coefficient of the artery A1. As described earlier, since the farther from an artery, the smaller the value of the damping coefficient β, as shown in FIG. 5, the farther from the artery A1, the smaller the magnitude (length) of the blood flow vector bv1.

Step S22: The specifying function 15b determines the blood flow strength coefficient and the damping coefficient of the second artery that is adjacent to the first artery and generates a second vector field related to the second artery. In the present step, for example, attention is focused on the second artery that is adjacent to the first artery among the plurality of arteries included in the medical image data acquired in the previous step S21 and a vector field of the second artery is generated. A specific generation method of the second vector field is the same as in the case of the first artery. In other words, first, the specifying function 15b determines the blood flow strength coefficient based on at least one of a diameter, a length, a position, the thickness of a wall, blood pressure, and blood flow of the second artery. Furthermore, the specifying function 15b determines the damping coefficient based on at least one of a distance from the second artery, a position of a thrombus, and properties of tissue surrounding the second artery. In addition, the specifying function 15b generates a second vector field (distance field) related to the second artery based on the determined blood flow strength coefficient and the determined damping coefficient.

An artery A2 on a right side of FIG. 5 is an example of the second artery. A plurality of blood flow vectors bv2 that radially spread from a center C2 of the artery A2 constitute the second vector field. Each blood flow vector bv2 has a direction of diffusion from the artery A2 and a magnitude in accordance with the blood flow strength coefficient and the damping coefficient of the artery A2. For example, the magnitude of the blood flow vector bv2 is proportional to a product of the blood flow strength coefficient and the damping coefficient. In a similar manner to the blood flow vector bv1, the farther from the artery A2, the smaller the blood flow vector bv2. In the example shown in FIG. 5, since the diameter of the artery A1 is larger than that of the artery A2, the blood flow strength coefficient of the artery A1 is larger than the blood flow strength coefficient of the artery A2. Therefore, when comparing positions at the same distance from the respective centers of the arteries A1 and A2, the magnitude of the blood flow vector bv1 is larger than the magnitude of the blood flow vector bv2.

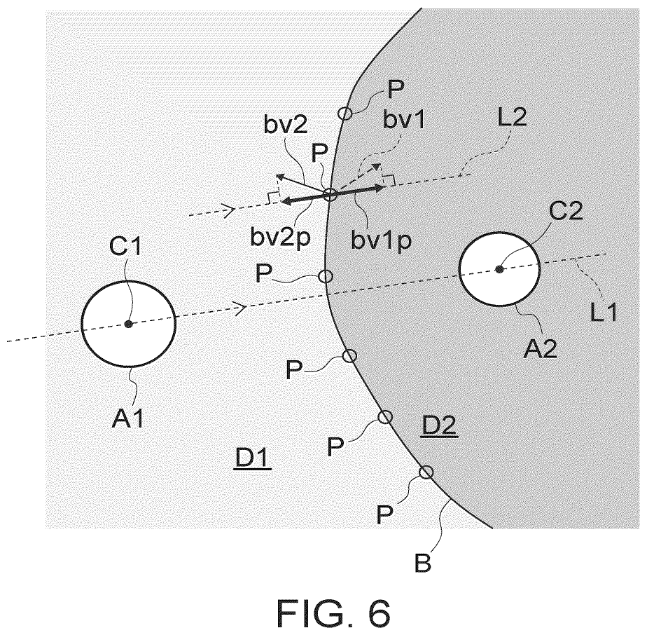

Step S23: The specifying function 15b demarcates a boundary B between a dominant area D1 of the artery A1 and a dominant area D2 of the artery A2 based on the first vector field and the second vector field generated in steps S21 and S22. For example, the specifying function 15b determines a plurality of points where projections of the blood flow vectors of the respective vector fields on a straight line parallel to a straight line that passes through the first artery and the second artery cancel each other out and connects the plurality of determined points. More specifically, as shown in FIG. 6, a straight line L2 parallel to a straight line L1 that passes through the center C1 of the artery A1 and the center C2 of the artery A2 is considered and a point on the straight line L2 is determined where a projection vector bv1p of the blood flow vector bv1 of the first vector field onto the straight line L2 and a projection vector bv2p of the blood flow vector bv2 of the second vector field onto the straight line L2 cancel each other out (in other words, a point where directions of the respective projection vectors are opposite but magnitudes of the projection vectors are the same and where a sum of the projection vectors is 0). Next, the straight line L2 is translated and a point P on the straight line L2 where a sum of projection vectors becomes 0 in a similar manner is determined. This is repeated to determine a plurality of points P. In FIG. 6, the sum of projection vectors becomes 0 at each point P. The specifying function 15b demarcates the boundary B between the dominant area D1 of the artery A1 and the dominant area D2 of the artery A2 by connecting the plurality of points P determined in this manner. Note that only representative points P are illustrated in FIG. 6.

Note that the straight line L1 is not limited to a line that passes through centers of the first and second arteries and may be a line that passes through a representative point such as a center of gravity of a cross-sectional shape of each artery. Moreover, the straight line L1 may be a straight line that passes through any point in the first artery and any point in the second artery.

In addition, a method of determining the points P is not limited to the method described above. For example, the specifying function 15b may generate a synthetic vector field by synthesizing the first vector field and the second vector field. In this case, the specifying function 15b may determine a plurality of vectors orthogonal to the straight line L1 in the synthetic vector field and demarcate the boundary B by connecting origins of the plurality of determined vectors.

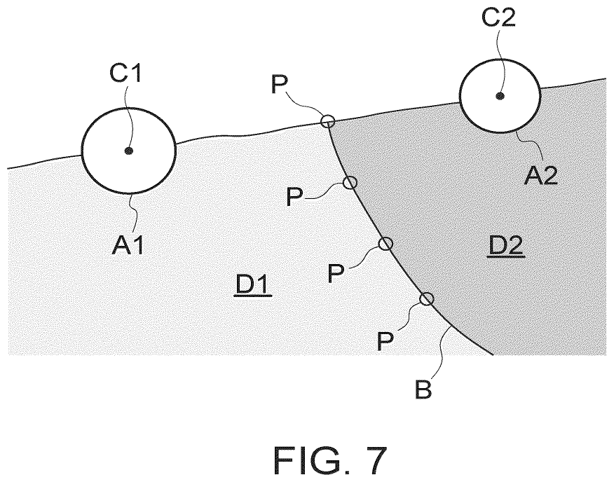

Furthermore, an object artery is not limited to arteries inside a tissue and may be arteries present on a surface of a tissue such as the myocardium. FIG. 7 shows an example of dominant areas D1 and D2 of the arteries A1 and A2 specified by the boundary B when the arteries are present on a surface of tissue (such as a case of coronary arteries of the myocardium).

Dominant areas are specified with respect to arteries other than the arteries A1 and A2 included in the medical image by executing steps S21 to S23. For example, when there is another artery adjacent to the artery A2 on a right side of the artery A2 in FIG. 6, after determining the blood flow strength coefficient and the damping coefficient of the other artery and generating a vector field, a boundary between the dominant area of the artery A2 and a dominant area of the other artery is determined based on the vector field and the second vector field related to the artery A2.

Let us return to the flowchart shown in FIG. 3 to continue the description thereof.

Step S3: The output function 15c outputs data (display data) for displaying the dominant areas of the first and second arteries specified in step S2. In the present embodiment, the output function 15c transmits the display data to the display 12. The display 12 displays an image created by superimposing and displaying the boundary B dividing the dominant areas D1 and D2 of the arteries A1 and A2 on a medical image in which the first and second arteries are drawn. In other words, the output function 15c outputs data for superimposing and displaying the boundary together with the first and second arteries on the medical image.

The medical image displayed on the display 12 may be a cross-sectional image of a three-dimensional tissue such as the heart or a two-dimensional image developed by equal-area map projection from a surface of the three-dimensional tissue. Equal-area map projection is a map projection in which a ratio between an area on the globe and a corresponding area on a map (in other words, an area scale) is the same anywhere. As equal-area map projection, various map projections such as Mollweide projection, sinusoidal projection, and Lambert equal-area projection can be used.

In addition, the boundary B is not limited to being displayed as a borderline as in FIG. 6 and may be indicated by displaying the dominant area D1 and the dominant area D2 in different display modes (colors, brightness, and the like).

According to the first embodiment described above, by taking the blood flow strength coefficient and the damping coefficient of each artery into consideration, the dominant area of each artery can be more accurately specified. Specifically, since the dominant area is specified in consideration of characteristics of blood vessels unique to each patient such as a blood flow, blood pressure, a blood vessel shape, a position, and the like of each artery, the dominant area of the artery can be accurately specified.

### Modification

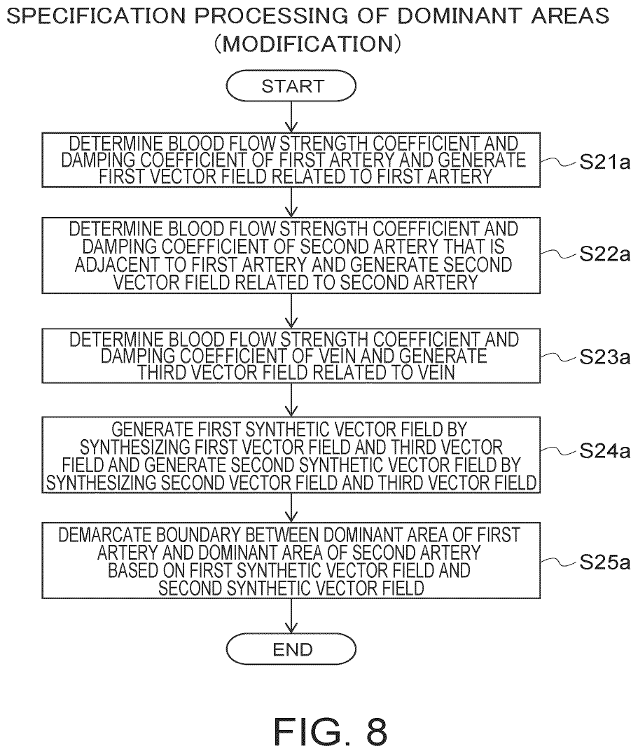

Next, a specification method of a dominant area according to a modification of the first embodiment will be described with reference to the flowchart in FIG. 8. In the present modification, the dominant area of each artery is specified in consideration of an impact exerted by a vein present in a periphery of the first artery and the second artery on blood flow. FIG. 8 shows a detailed flow of the processing in step S2.

Step S21a: The specifying function 15b determines the blood flow strength coefficient and the damping coefficient of the first artery and generates a first vector field related to the first artery. Since contents of the present step are the same as step S21 described earlier, a detailed description will be omitted.

Step S22a: The specifying function 15b determines the blood flow strength coefficient and the damping coefficient of the second artery that is adjacent to the first artery and generates a second vector field related to the second artery. Since contents of the present step are the same as step S22 described earlier, a detailed description will be omitted.

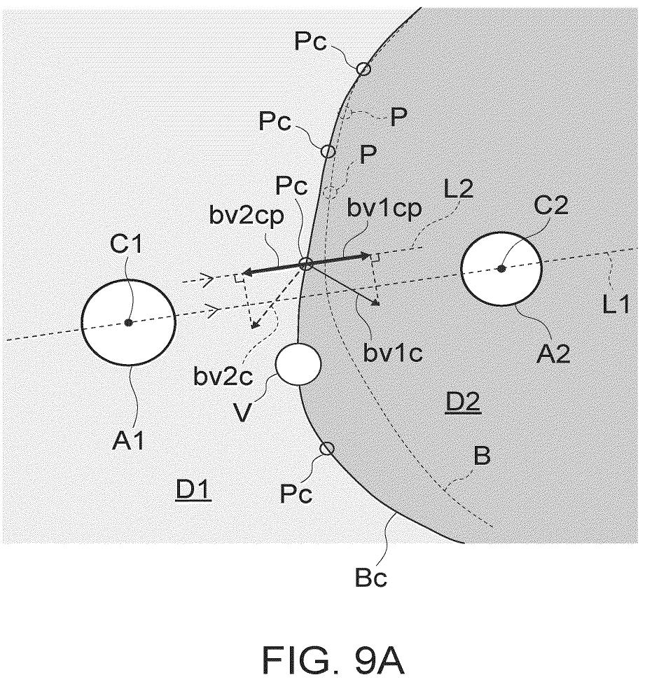

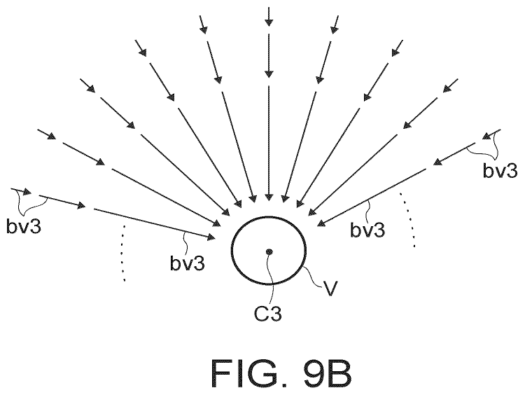

Step S23a: The specifying function 15b determines the blood flow strength coefficient and the damping coefficient of a vein included in the site of the medical image data and generates a third vector field related to the vein. For example, as shown in FIG. 9A, the vein is a vein V present between the artery A1 and the artery A2.

A blood flow vector constituting the third vector field related to the vein has a direction that converges on the vein and a magnitude in accordance with the blood flow strength coefficient and the damping coefficient of the vein. The blood flow strength coefficient of the vein is a coefficient that represents a strength of a blood flow of blood that converges from outside to inside of the vein and the damping coefficient of the vein is a coefficient that represents damping characteristics of a blood flow in a peripheral tissue (such as the myocardium) of the vein. The blood flow strength coefficient and the damping coefficient of the vein are determined in the same manner as in the case of arteries. Specifically, the specifying function 15b determines the blood flow strength coefficient based on at least one of a vascular diameter, a vascular length, a vascular position, a thickness of a vascular wall, blood pressure, and blood flow of the vein and determines the damping coefficient based on at least one of a distance from the vein, a position of a thrombus in the vein, and properties of tissue surrounding the vein.

FIG. 9B shows an example of a vector field constituted of blood flow vectors of a vein. A plurality of blood flow vectors bv3 that converge toward a center C3 of the vein V constitute the third vector field. Each blood flow vector bv3 has a direction of convergence toward the vein V and a magnitude in accordance with the blood flow strength coefficient and the damping coefficient of the vein V. As shown in FIG. 9B, the farther from the vein V, the smaller the magnitude (length) of the blood flow vector bv3.

Step S24a: The specifying function 15b generates a first synthetic vector field by synthesizing the first vector field and the third vector field. In addition, the specifying function 15b generates a second synthetic vector field by synthesizing the second vector field and the third vector field.

Step S25a: The specifying function 15b demarcates a boundary between a dominant area of the first artery and a dominant area of the second artery based on the first synthetic vector field and the second synthetic vector field. The demarcation of the boundary can be performed using a similar method to step S23 described above. For example, the specifying function 15b demarcates the boundary by determining a plurality of points where projections of the blood flow vectors of the respective synthetic vector fields on a straight line parallel to a straight line that passes through the first artery and the second artery cancel each other out and connecting the plurality of determined points.

More specifically, as shown in FIG. 9A, a straight line L2 parallel to a straight line L1 that passes through the center C1 of the artery A1 and the center C2 of the artery A2 is considered and a point is determined where a projection vector bv1cp of a blood flow vector bv1c of the first synthetic vector field onto the straight line L2 and a projection vector bv2cp of a blood flow vector bv2c of the second synthetic vector field onto the straight line L2 cancel each other out. In FIG. 9A, the sum of projection vectors becomes 0 at each point Pc. By connecting the plurality of points Pc determined in this manner, the specifying function 15b demarcates a boundary Bc between the dominant area D1 of the artery A1 and the dominant area D2 of the artery A2 in a case where the vein V is present. Note that a boundary B in FIG. 9A represents a boundary in a case where the vein V is not present.

According to the present modification, a dominant area of an artery can be specified in consideration of a presence of a vein as described above. In the example in FIG. 9A, since the vein V is present on a side of the artery A1, the boundary has shifted toward the side of the artery A1 as compared to a case where the vein V is absent.

Note that a method of determining the points Pc is not limited to the method described above. For example, the specifying function 15b may demarcate the boundary Bc by generating a third synthetic vector field by synthesizing the first synthetic vector field and the second synthetic vector field, determining a plurality of vectors orthogonal to the straight line L1 in the third synthetic vector field, and connecting origins of the plurality of determined vectors.

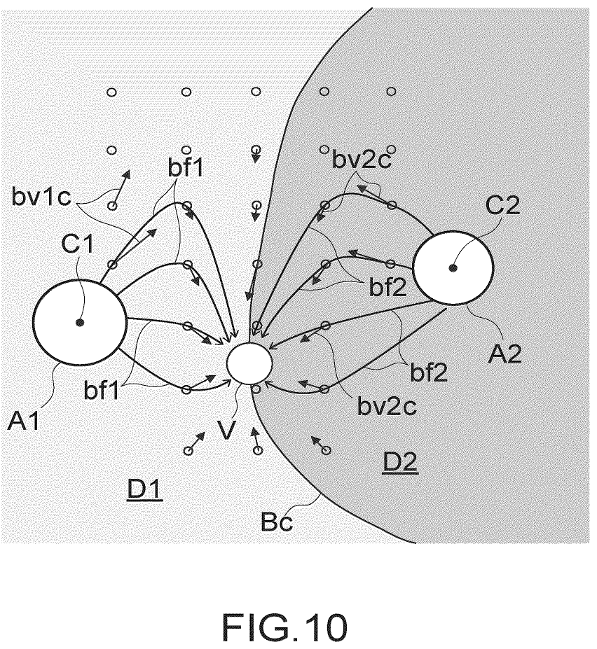

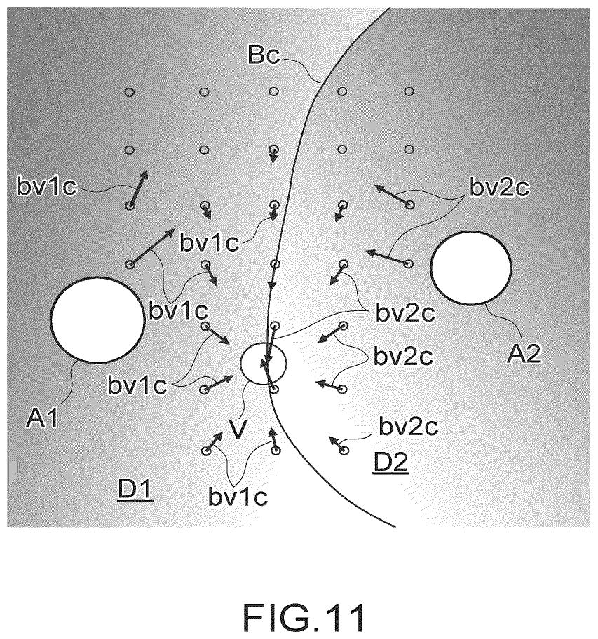

In addition, as shown in FIG. 10, blood flows bf1 and bf2 based on blood flow vectors may be displayed on a medical image displaying the specified dominant area of each artery. Circular marks in FIG. 10 denote only representative points in peripheral areas of arteries (a similar description also applies to FIG. 11). The output function 15c outputs data for displaying a blood flow from the artery A1 toward the vein V based on the first synthetic vector field and outputs data for displaying a blood flow from the artery A2 toward the vein V based on the second synthetic vector field. Accordingly, the blood flow bf1 constructed by connecting blood flow vectors bv1c of the first synthetic vector field is displayed and the blood flow bf2 constructed by connecting blood flow vectors bv2c of the second synthetic vector field is displayed. The blood flows bf1 and bf2 are discharged from the arteries A1 and A2 and absorbed by the vein V. Alternatively, only one of the blood flow bf1 and the blood flow bf2 may be displayed. A blood flow may be determined by connecting blood flow vectors of respective data points (voxels) of medical image data.

In addition, lines representing the blood flows bf1 and bf2 may be displayed by gradation. For example, a line representing a blood flow may be displayed by gradation so that a side of an artery is colored in a first color (for example, red) and a side of a vein is colored in a second color (for example, blue).

In addition, as shown in FIG. 11, each dominant area may be displayed by gradation in a medical image displaying the specified dominant area of each artery. In this case, the output function 15c outputs data for displaying the dominant area of the first artery and the dominant area of the second artery by gradation so that a side of an artery of the dominant areas is colored in a first color and a side of a vein of the dominant areas is colored in a second color. Accordingly, as shown in FIG. 11, the dominant area D1 of the first artery A1 and the dominant area D2 of the second artery A2 are displayed by gradation so that a side of the arteries is colored in a first color (for example, red) and a side of a vein is colored in a second color (for example, blue).

Note that the blood flow vectors bv1c and bv2c need not be displayed in FIGS. 10 and 11. A user may be enabled to designate, via the input interface 13 of the medical image processing apparatus 1, whether or not to display blood flow vectors and which blood flow from which artery is to be displayed.

### Example of Application

Next, as a specific example of application of the medical image processing apparatus and method described above, an application to the specification of a dominant area of a coronary artery of the heart will be described.

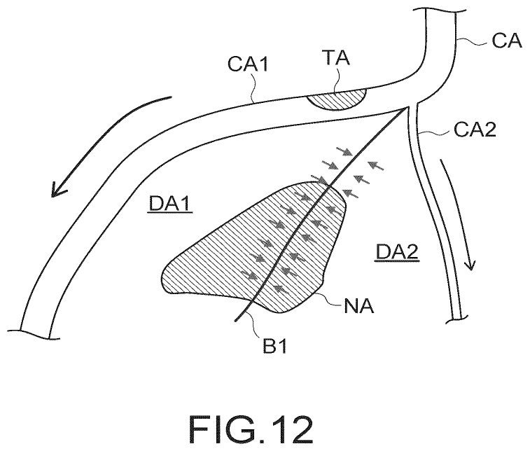

FIG. 12 shows an example of a display image in which a heart surface is developed into a two-dimensional image by equal-area map projection. In the diagram, a plurality of arrows oriented toward a boundary B1 represent blood flow vectors. The arrows need not be displayed. As shown in FIG. 12, in this example, a coronary artery CA branches into a coronary artery CA1 and a coronary artery CA2, with the coronary artery CA1 being thicker than the coronary artery CA2. Since the coronary artery CA1 is constricted by a thrombus TA, a blood flow is reduced as compared to a normal state. In addition, a necrotic area NA of the myocardium is present between the coronary artery CA1 and the coronary artery CA2. A boundary B1 represents a boundary determined by the specification method of a dominant area according to the embodiment described earlier and is a boundary between a dominant area DA1 of the coronary artery CA1 and a dominant area DA2 of the coronary artery CA2. As shown in FIG. 12, the boundary B1 straddles the necrotic area NA. The boundary B1 can be used as a resection line that reduces a volume of hemorrhage when performing a resection of the necrotic area NA.

FIG. 13 shows an example of a display image in a case where the thrombus TA is virtually removed in a simulation. In the diagram, a plurality of arrows oriented toward a boundary B2 represent blood flow vectors. The arrows need not be displayed. In this example, assuming that the thrombus TA has been removed, a dominant area is specified based on the blood flow strength coefficient and the damping coefficient in a normal state of the coronary artery CA1. In FIG. 13, the specified boundary B2 between the dominant areas is superimposed and displayed on an image of a myocardium. The boundary B2 has shifted toward a side of the coronary artery CA2 and the necrotic area NA is now included in the dominant area DA1 of the coronary artery CA1. From this result, a user such as a physician can predict that the entire necrotic area NA will belong to the dominant area of the coronary artery CA due to removal of the thrombus TA. In addition, when there are a plurality of thrombi in an artery, performing a plurality of simulations while changing the thrombus to be removed enables the user such as a physician to study what kind of vascular treatment is to be effective.

The boundary B1 prior to treatment can be displayed so as to be distinguishable from the boundary B2 as shown in FIG. 13. Accordingly, a change in boundaries due to the treatment can be readily comprehended.

In addition to the removal of a thrombus, a change in a dominant area of an artery adjacent to a blood vessel can be simulated before performing a treatment involving tying up or resecting the blood vessel.

In addition, images of FIGS. 12 and 13 may be simultaneously displayed on the display 12 in order to compare a dominant area prior to treatment and a dominant area based on a simulation result (a dominant area after a virtual treatment) with each other. Accordingly, the user such as a physician can readily comprehend a predicted effect of a treatment in advance.

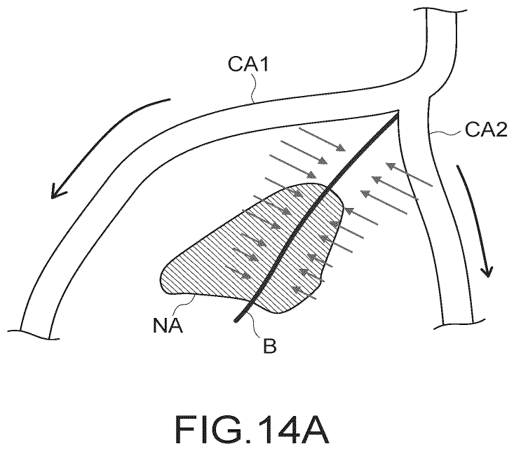

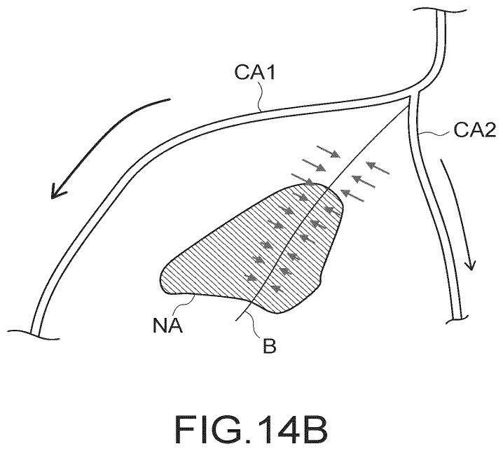

As shown in FIGS. 14A and 14B, a display mode of a boundary between dominant areas may be changed according to a strength of a blood flow that collides with the boundary. For example, when both the coronary artery CA1 and the coronary artery CA2 are thick and have large blood flows, a relatively thick boundary B may be displayed as shown in FIG. 14A. On the other hand, when both the coronary artery CA1 and the coronary artery CA2 are thin, a thin boundary B may be displayed as shown in FIG. 14B. The display mode to be changed is not limited to the thickness of the boundary B and may be a color, a line style, or the like of the boundary B.

In this manner, the output function 15c may change a display mode of a boundary according to a magnitude of a blood flow oriented toward the boundary. Accordingly, the user such as a physician can readily comprehend a strength of a blood flow from the display mode of the boundary. For example, while blood flow vectors (arrows oriented toward the boundary B) in FIGS. 14A and 14B may be hidden in order to avoid an overcomplicated screen display, the strength of a blood flow can be readily comprehended from the display mode of the boundary even in this case.

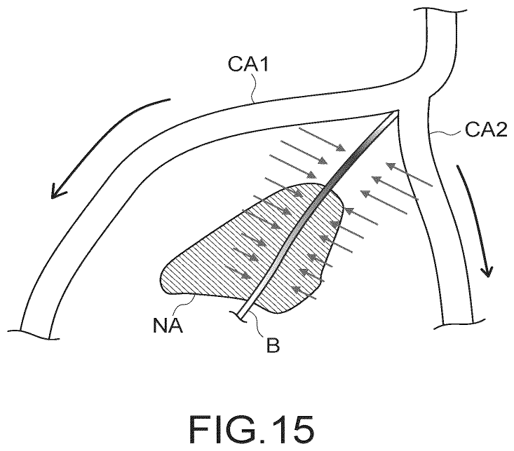

As shown in FIG. 15, a boundary may be displayed by gradation in accordance with a strength of a blood flow. In other words, the boundary B may be displayed by gradation along the boundary B in accordance with a magnitude of a blood flow oriented toward the boundary. For example, the boundary B is displayed by gradation so that a portion in which the strength of the blood flow is highest is colored in a first color (for example, dark red) and a portion in which the strength of the blood flow is lowest is colored in a second color (for example, pale red). Accordingly, the user such as a physician can readily comprehend a portion where the blood flow is strong (or weak) from the gradation of the boundary. For example, while blood flow vectors (arrows oriented toward the boundary B) in FIG. 15 may be hidden in order to avoid an overcomplicated screen display, the strength of a blood flow in each portion of the boundary can be readily comprehended from the gradation of the boundary even in this case.

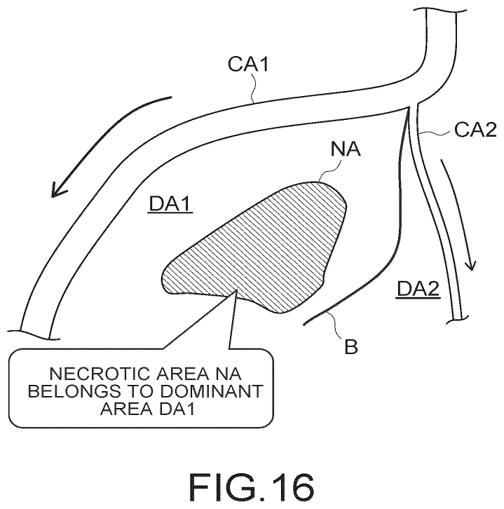

Note that the output function 15c may output data for indicating which dominant area among dominant areas specified by the specifying function 15b a tissue of which properties are not normal such as a tissue hardened due to advanced ischemia, a necrotic area, or an affected area belongs to. In the example in FIG. 16, the fact that the necrotic area NA belongs to the dominant area DA1 is indicated by a pop-up display. Accordingly, the user such as a physician can readily comprehend which dominant area of an artery a tissue of which properties are not normal belongs to. Note that the display mode is not limited to a pop-up format.

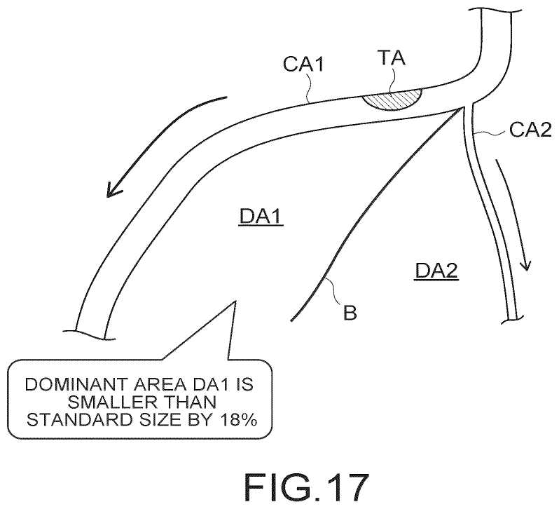

In addition, when a size of a dominant area specified by the specifying function 15b differs from a standard size by a predetermined value or more, the output function 15c may output data for indicating information to that effect. In the example shown in FIG. 17, the fact that the size of the dominant area DA1 is smaller by 18% than a standard size due to the thrombus TA in the coronary artery CA1 is indicated by a pop-up display. Accordingly, the user such as a physician can readily comprehend a dominant area of which the size differs from a standard size by a predetermined value or more. Note that the display mode is not limited to a pop-up format.

As described above, in the first embodiment, since dominant areas of a plurality of arteries are specified by taking the blood flow strength coefficient and the damping coefficient of the plurality of arteries into consideration, the dominant area of each artery can be more accurately specified. Accordingly, for example, an accurate simulation related to reperfusion can be performed and a resection line that reduces a volume of hemorrhage when performing a resection of a necrotic area of the myocardium can be determined.

### Second Embodiment

In the first embodiment, when a first artery and a second artery are present, a dominant area of each artery is specified. In the second embodiment, when an artery and a vein are present, a dominant area of the artery is specified. Since a configuration of the medical image processing apparatus according to the second embodiment is the same as the configuration of the medical image processing apparatus 1 described in the first embodiment, a description thereof will be omitted.

### <Medical Image Processing Method (Second Embodiment)>

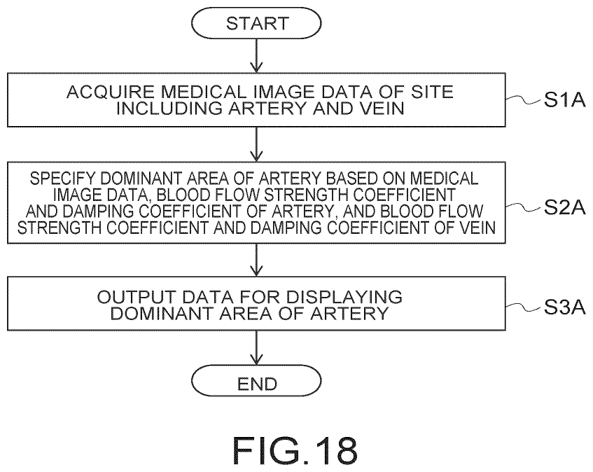

An example of a medical image processing method according to the second embodiment will be described with reference to the flowchart in FIG. 18.

Step S1A: The acquiring function 15a acquires medical image data of a site including an artery and a vein adjacent to the artery. Details of an acquisition method of the medical image data is the same as in step S1 described earlier.

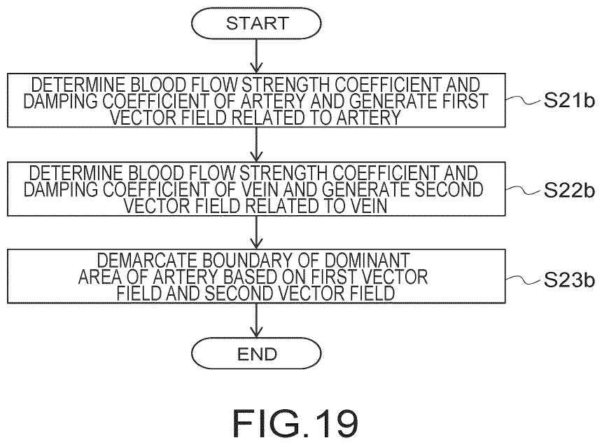

Step S2A: The specifying function 15b specifies a dominant area of the artery based on the medical image data acquired in step S1A, a blood flow strength coefficient and a damping coefficient of the artery, and a blood flow strength coefficient and a damping coefficient of the vein. Details of the present step will be described with reference to the flowchart in FIG. 19.

Step S21b: The specifying function 15b determines the blood flow strength coefficient and the damping coefficient of the artery and generates a first vector field related to the artery. Details of the present step are the same as in step S21 described earlier.

Step S22b: The specifying function 15b determines the blood flow strength coefficient and the damping coefficient of the vein and generates a second vector field related to the vein. A specific generation method of the second vector field is the same as in step S23a described earlier.

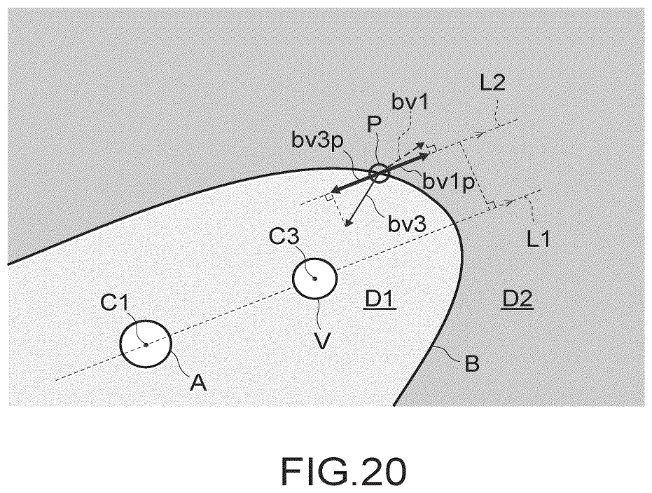

Step S23b: The specifying function 15b demarcates a boundary of the dominant area of the artery based on the first vector field and the second vector field generated in steps S21b and S22b. For example, the specifying function 15b determines a plurality of points where projections of the blood flow vectors of the respective vector fields on a straight line parallel to a straight line that passes through the artery and the vein cancel each other out and connects the plurality of determined points. More specifically, as shown in FIG. 20, a straight line L2 parallel to a straight line L1 that passes through a center C1 of an artery A and a center C3 of a vein V is considered and a point P is determined where a projection vector bv1p of a blood flow vector bv1 of the first vector field onto the straight line L2 and a projection vector bv3p of the blood flow vector bv3 of the second vector field onto the straight line L2 cancel each other out. Next, the straight line L2 is translated and a point on the straight line L2 where a sum of projection vectors becomes 0 is determined in a similar manner. This is repeated to determine a plurality of points. The specifying function 15b demarcates the boundary B of the dominant area D1 of the artery A by connecting the plurality of points determined in this manner.

Let us return to the flowchart shown in FIG. 18 to continue the description thereof.

Step S3A: The output function 15c outputs data (display data) for displaying the dominant area of the artery specified in step S2A. Details of the present step are the same as in step S3 described earlier.

The output function 15c may output data for displaying a blood flow from the artery toward the vein based on the first and second vector fields. Accordingly, a screen display similar to that according to the modification of the first embodiment can be performed. In addition, lines representing blood flows may be displayed by gradation. Furthermore, the output function 15c may output data for displaying the dominant area of the artery by gradation so that a side of the artery of the dominant area is colored in a first color and a side of the vein of the dominant area is colored in a second color.

As described above, in the second embodiment, since a dominant area of an artery is specified by taking the blood flow strength coefficient and the damping coefficient of the artery and the blood flow strength coefficient and the damping coefficient of a vein into consideration, the dominant area of the artery can be more accurately specified.

### Third Embodiment

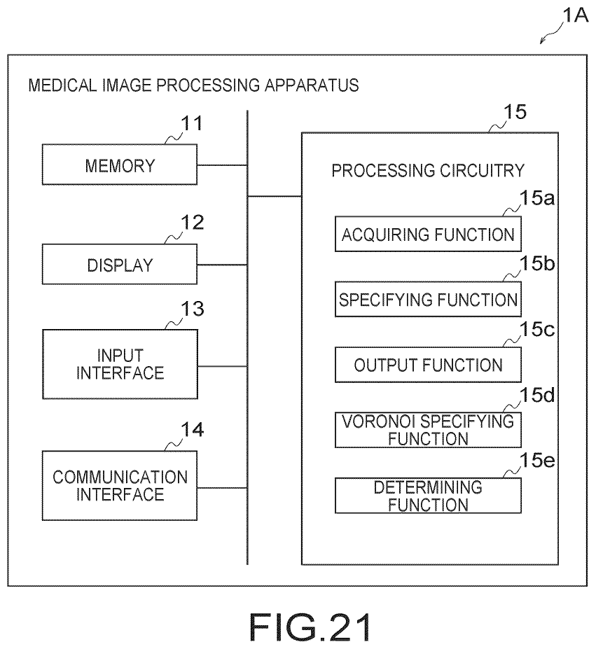

Next, a medical image processing apparatus according to the third embodiment will be described. FIG. 21 is a block diagram showing an example of a configuration of a medical image processing apparatus 1A according to the present embodiment. In FIG. 21, constituent elements with the same functions as those in FIG. 1 will be denoted by same reference signs.

In addition to the respective functions (the acquiring function 15a, the specifying function 15b, and the output function 15c) of the processing circuitry 15 described in the first embodiment, the medical image processing apparatus 1A includes a Voronoi specifying function 15d and a determining function 15e. The Voronoi specifying function 15d is an example of a Voronoi specifier and the determining function 15e is an example of a determiner.

In the present embodiment, the respective processing functions executed by the acquiring function 15a, the specifying function 15b, the output function 15c, the Voronoi specifying function 15d, and the determining function 15e are stored in the memory 11 in the form of programs that can be executed by a computer. Specifically, the processing circuitry 15 is constituted of a processor and realizes a function corresponding to each program by reading and executing the program from the memory 11. In other words, the processing circuitry 15 in a state of having read each program is to include each function shown in the processing circuitry 15 in FIG. 21.

The Voronoi specifying function 15d specifies a dominant area of a blood vessel by Voronoi tessellation. Specifically, the Voronoi specifying function 15d specifies a dominant area of a first blood vessel and a dominant area of a second blood vessel by Voronoi tessellation based on shape information of the first blood vessel and shape information of the second blood vessel included in medical image data.

The determining function 15e determines, based on predetermined conditions, which of the specifying function 15b and the Voronoi specifying function 15d is to be used to specify a dominant area. The predetermined conditions include a required specification accuracy, mechanical specifications (processing speed and the like) of the medical image processing apparatus, and urgency of an operation or an examination.

### <Medical Image Processing Method (Third Embodiment)>

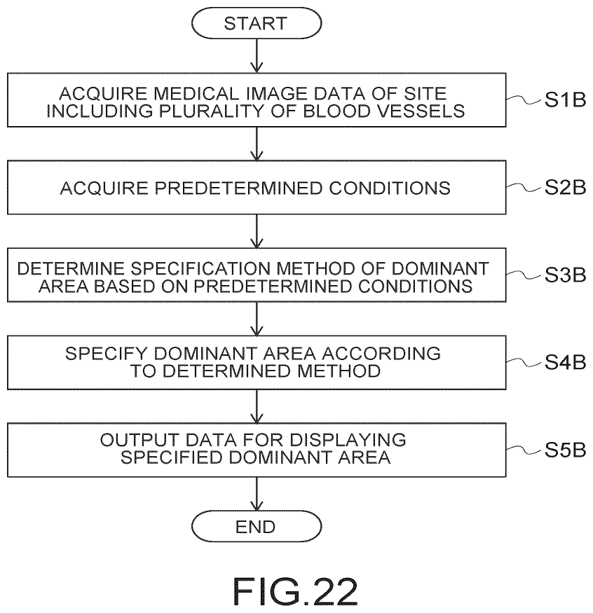

An example of a medical image processing method using the medical image processing apparatus 1A described above will be described with reference to the flowchart in FIG. 22.

Step S1B: The acquiring function 15a acquires medical image data of a site including a plurality of blood vessels. Details of an acquisition method of the medical image data is the same as in step S1 described earlier.

Step S2B: The acquiring function 15a acquires predetermined conditions for determining a specification method. For example, the acquiring function 15a acquires a required specification accuracy, urgency, and the like inputted by the user via the input interface 13 as the predetermined conditions. Note that the acquiring function 15a may acquire information related to the mechanical specifications of the medical image processing apparatus 1A as the predetermined conditions from an operating system of the medical image processing apparatus 1A or the like. Alternatively, the acquiring function 15a may acquire information indicating urgency as the predetermined conditions from an electronic health record of a patient or the like.

Step S3B: The determining function 15e determines the specification method of a dominant area based on the predetermined conditions acquired in step S2B. For example, the determining function 15e determines to perform specification using the Voronoi specifying function 15d when the accuracy (specification accuracy) required of demarcation of the dominant area is low, when the mechanical specifications of the medical image processing apparatus 1A is low, or when urgency is high.

Step S4B: The specifying function 15b or the Voronoi specifying function 15d specifies a dominant area according to the method determined in step S3B. In other words, when the determining function 15e determines to perform specification using the specifying function 15b, step S2 or step S2A described earlier is performed in the present step. On the other hand, when the determining function 15e determines to perform specification using the Voronoi specifying function 15d, the Voronoi specifying function 15d specifies a dominant area by Voronoi tessellation.

Step S5B: The output function 15c outputs data for displaying the dominant area specified in step S4B. Based on the data, the display 12 displays an image created by superimposing and displaying a boundary of the dominant area of the artery on a medical image in which the artery is drawn.

According to the third embodiment, a specification method of a dominant area by Voronoi tessellation and a specification method of a dominant area that takes blood vessel characteristics unique to a patient into consideration can be appropriately used based on conditions such as a required specification accuracy.

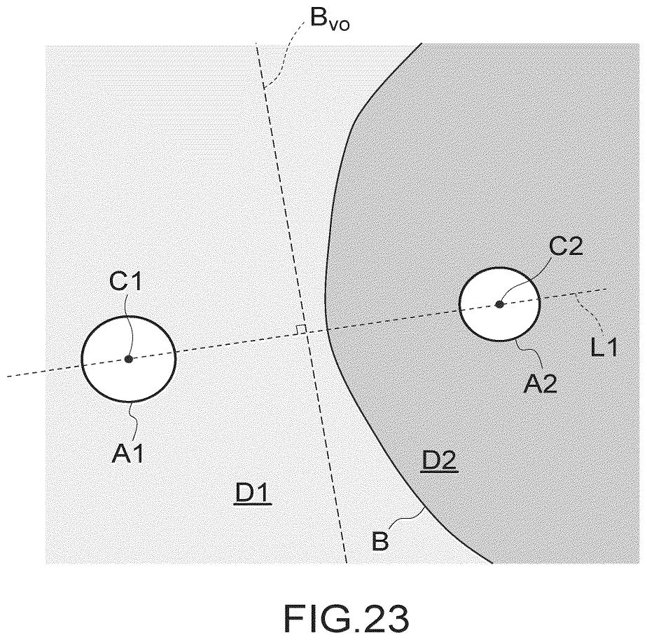

Note that both a specification of a dominant area by the specifying function 15b and a specification of a dominant area by the Voronoi specifying function 15d may be performed. In this case, in addition to data for displaying the dominant area specified by the specifying function 15b, the output function 15c may output data for displaying the dominant area specified by the Voronoi specifying function 15d. FIG. 23 represents a screen example on which both a boundary B demarcated by the specifying function 15b and a boundary Bvo demarcated by the Voronoi specifying function 15d are displayed. The boundary B and the boundary Bvo may be made to be distinguishable from each other by changing display modes such as a line color or a line style.

The medical image processing method described above is simply an example and various modifications can be made thereto. For example, steps S2B and S3B can be performed before step S1B.

For example, the term “processor” as used in the description given above means a CPU (Central Processing Unit), a GPU (Graphics Processing Unit), or circuitry such as an Application Specific Integrated Circuit (ASIC) or a Programmable Logic Device (for example, a Simple Programmable Logic Device (SPLD), a Complex Programmable Logic Device (CPLD), or a Field Programmable Gate Array (FPGA)). The processor realizes functions by reading and executing a program stored in the memory 11. Note that a configuration may be adopted in which a program is directly incorporated into the circuitry of the processor instead of being stored in the memory 11. In this case, the processor realizes functions by reading and executing the program incorporated into the circuitry. The processor is not limited to a single circuit configuration of the processor and a plurality of independent circuits may be combined to constitute a single processor and the functions may be realized by the single processor. Furthermore, the plurality of constituent elements in FIG. 1 may be integrated into a single processor and the functions may be realized by the single processor.

The medical image processing methods described with reference to FIGS. 3, 4, 8, 18, 19, and 22 can be realized by having a computer such as a personal computer or a workstation execute a medical image processing program prepared in advance. The medical image processing program can be distributed via a network such as the Internet. Alternatively, the medical image processing program can be recorded on a non-transitory computer-readable recording medium such as a hard disk, a flexible disk (FD), a CD-ROM, an MO, or a DVD and can be executed by being read from the recording medium by a computer.

Although several embodiments have been described above, these embodiments have been presented only as examples, and are not intended to limit the scope of the invention. The novel devices and methods described herein can be implemented in a variety of other forms. In addition, various omissions, substitutions, and changes can be made to the forms of the apparatus and the method described in the present specification without departing from the gist of the invention. The appended claims and their equivalents are intended to include such forms and modifications as fall within the scope and spirit of the invention.

1. A medical image processing apparatus comprising:
processing circuitry configured to
acquire medical image data of a site including a first blood vessel that is an artery and a second blood vessel that is an artery or a vein;
specify a dominant area of the first blood vessel based on the medical image data, a blood flow strength coefficient and a damping coefficient of the first blood vessel, and a blood flow strength coefficient and a damping coefficient of the second blood vessel; and
output data for displaying the specified dominant area.

2. The medical image processing apparatus of claim 1, wherein
the blood flow strength coefficient of the first blood vessel is a coefficient based on at least one of a vascular diameter, a vascular length, a vascular position, a thickness of a vascular wall, blood pressure, and blood flow of the first blood vessel and the damping coefficient of the first blood vessel is a coefficient based on at least one of a distance from the first blood vessel, a position of a thrombus in the first blood vessel, and properties of tissue surrounding the first blood vessel,
the blood flow strength coefficient of the second blood vessel is a coefficient based on at least one of a vascular diameter, a vascular length, a vascular position, a thickness of a vascular wall, blood pressure, and blood flow of the second blood vessel and the damping coefficient of the second blood vessel is a coefficient based on at least one of a distance from the second blood vessel, a position of a thrombus in the second blood vessel, and properties of tissue surrounding the second blood vessel.

3. The medical image processing apparatus of claim 1, wherein
the second blood vessel is an artery adjacent to the first blood vessel,
the blood flow strength coefficient of the first blood vessel represents a strength of a blood flow of blood diffused from inside to outside of the first blood vessel and the damping coefficient of the first blood vessel represents damping characteristics of the blood flow in a peripheral tissue of the first blood vessel,
the blood flow strength coefficient of the second blood vessel represents a strength of a blood flow of blood diffused from inside to outside of the second blood vessel and the damping coefficient of the second blood vessel represents damping characteristics of the blood flow in a peripheral tissue of the second blood vessel,
wherein the processing circuitry is further configured to
specify dominant areas of the first blood vessel and the second blood vessel; and
output data for displaying the dominant areas of the first blood vessel and the second blood vessel.

4. The medical image processing apparatus of claim 3, wherein the processing circuitry is further configured to
generate a first vector field with respect to the first blood vessel, wherein a blood flow vector constituting the first vector field has a direction of diffusion from the first blood vessel and a magnitude in accordance with the blood flow strength coefficient and the damping coefficient of the first blood vessel;
generate a second vector field with respect to the second blood vessel, wherein a blood flow vector constituting the second vector field has a direction of diffusion from the second blood vessel and a magnitude in accordance with the blood flow strength coefficient and the damping coefficient of the second blood vessel; and
demarcate a boundary between the dominant area of the first blood vessel and the dominant area of the second blood vessel based on the first vector field and the second vector field.

5. The medical image processing apparatus of claim 3, wherein
the site of the medical image data includes a third blood vessel that is a vein,
a blood flow strength coefficient of the third blood vessel represents a strength of a blood flow of blood that converges from outside to inside of the third blood vessel and the damping coefficient of the third blood vessel represents damping characteristics of the blood flow in a peripheral tissue of the third blood vessel,
wherein the processing circuitry is further configured to
specify dominant areas of the first blood vessel and the second blood vessel based on the medical image data and blood flow strength coefficients and damping coefficients of the first to third blood vessels; and
output data for displaying the dominant areas of the first blood vessel and the second blood vessel.

6. The medical image processing apparatus of claim 5, wherein the processing circuitry is further configured to
generate a first vector field with respect to the first blood vessel, wherein a blood flow vector constituting the first vector field has a direction of diffusion from the first blood vessel and a magnitude in accordance with the blood flow strength coefficient and the damping coefficient of the first blood vessel;
generate a second vector field with respect to the second blood vessel, wherein a blood flow vector constituting the second vector field has a direction of diffusion from the second blood vessel and a magnitude in accordance with the blood flow strength coefficient and the damping coefficient of the second blood vessel;
generate a third vector field with respect to the third blood vessel, wherein a blood flow vector constituting the third vector field has a direction of convergence toward the third blood vessel and a magnitude in accordance with the blood flow strength coefficient and the damping coefficient of the third blood vessel;
generate a first synthetic vector field by synthesizing the first vector field and the third vector field and generate a second synthetic vector field by synthesizing the second vector field and the third vector field; and
demarcate a boundary between the dominant area of the first blood vessel and the dominant area of the second blood vessel based on the first synthetic vector field and the second synthetic vector field.

7. The medical image processing apparatus of claim 6, wherein the processing circuitry is further configured to
output data for displaying a blood flow from the first blood vessel toward the third blood vessel based on the first synthetic vector field and/or output data for displaying a blood flow from the second blood vessel toward the third blood vessel based on the second synthetic vector field.

8. The medical image processing apparatus of claim 7, wherein
a line representing the blood flow is displayed by gradation so that a side of an artery is colored in a first color and a side of a vein is colored in a second color.

9. The medical image processing apparatus of claim 6, wherein the processing circuitry is further configured to
output data for displaying the dominant area of the first blood vessel and the dominant area of the second blood vessel by gradation so that a side of an artery is colored in a first color and a side of a vein is colored in a second color.

10. The medical image processing apparatus of claim 1, wherein
the second blood vessel is a vein adjacent to the first blood vessel,
the blood flow strength coefficient of the first blood vessel represents a strength of a blood flow of blood diffused from inside to outside of the first blood vessel and the damping coefficient of the first blood vessel represents damping characteristics of the blood flow in a peripheral tissue of the first blood vessel,
the blood flow strength coefficient of the second blood vessel represents a strength of a blood flow of blood that converges from outside to inside of the second blood vessel and the damping coefficient of the second blood vessel represents damping characteristics of the blood flow in a peripheral tissue of the second blood vessel,
wherein the processing circuitry is further configured to
specify a dominant area of the first blood vessel; and
output data for displaying the dominant area of the first blood vessel.

11. The medical image processing apparatus of claim 10, wherein the processing circuitry is further configured to
generate a first vector field with respect to the first blood vessel, wherein a blood flow vector constituting the first vector field has a direction of diffusion from the first blood vessel and a magnitude in accordance with the blood flow strength coefficient and the damping coefficient of the first blood vessel;
generate a second vector field with respect to the second blood vessel, wherein a blood flow vector constituting the second vector field has a direction of convergence toward the second blood vessel and a magnitude in accordance with the blood flow strength coefficient and the damping coefficient of the second blood vessel; and
demarcate a boundary of the dominant area of the first blood vessel based on the first vector field and the second vector field.

12. The medical image processing apparatus of claim 1, wherein the processing circuitry is further configured to
output data for displaying the boundary of the dominant area together with the first and second blood vessels, wherein a display mode of the boundary changes according to a magnitude of a blood flow oriented toward the boundary.

13. The medical image processing apparatus of claim 1, wherein the processing circuitry is further configured to
output data for displaying the boundary of the dominant area together with the first and second blood vessels, wherein the boundary is displayed by gradation along the boundary according to a magnitude of a blood flow oriented toward the boundary.

14. The medical image processing apparatus of claim 1, wherein the processing circuitry is further configured to
specify a first dominant area of the first blood vessel based on the blood flow strength coefficient and the damping coefficient of the first blood vessel prior to treatment;
specify a second dominant area of the first blood vessel based on the blood flow strength coefficient and the damping coefficient of the first blood vessel after a virtual treatment; and
output data for comparably displaying the first and second dominant areas.

15. The medical image processing apparatus of claim 1, wherein the processing circuitry is further configured to
output data for indicating which dominant area among the specified dominant areas a tissue of which properties differ from normal belongs to.

16. The medical image processing apparatus of claim 1, wherein the processing circuitry is further configured to
output, when a size of the specified dominant area differs from a standard size by a predetermined value or more, data for indicating information to that effect.

17. The medical image processing apparatus of claim 1, wherein
the second blood vessel is an artery adjacent to the first blood vessel,
wherein the processing circuitry is further configured to
determine which of a first specification method and a second specification method is to be used to perform specification of dominant areas based on predetermined conditions, wherein the first specification method is a method of specifying a dominant area of the first blood vessel and a dominant area of the second blood vessel based on the medical image data, the blood flow strength coefficient and the damping coefficient of the first blood vessel, and the blood flow strength coefficient and the damping coefficient of the second blood vessel, and the second specification method is a method of specifying a dominant area of the first blood vessel and a dominant area of the second blood vessel by Voronoi tessellation based on shape information of the first blood vessel and shape information of the second blood vessel; and
when it is determined that specification of dominant areas by the second specification method is to be performed, specify the dominant areas of the first and second blood vessels by the second specification method and output data for displaying the dominant areas specified by the second specification method in place of data for displaying the dominant areas specified by the first specification method.

18. The medical image processing apparatus of claim 1, wherein
the second blood vessel is an artery adjacent to the first blood vessel,
wherein the processing circuitry is further configured to
specify a dominant area of the first blood vessel and a dominant area of the second blood vessel by Voronoi tessellation based on shape information of the first blood vessel and shape information of the second blood vessel; and
output data for displaying the specified dominant areas.

19. A medical image processing method comprising:
acquiring medical image data of a site including a first blood vessel that is an artery and a second blood vessel that is an artery or a vein,
specifying a dominant area of the first blood vessel based on the medical image data, a blood flow strength coefficient and a damping coefficient of the first blood vessel, and a blood flow strength coefficient and a damping coefficient of the second blood vessel, and
outputting data for displaying the specified dominant areas.

20. A non-transitory computer-readable storage medium storing a program for causing a computer to execute a process, comprising:
acquiring medical image data of a site including a first blood vessel that is an artery and a second blood vessel that is an artery or a vein,
specifying a dominant area of the first blood vessel based on the medical image data, a blood flow strength coefficient and a damping coefficient of the first blood vessel, and a blood flow strength coefficient and a damping coefficient of the second blood vessel, and
outputting data for displaying the specified dominant areas.
